(19) United States
(12) Patent Application Publication
(10) Pub. No.: US 2022/0169991 A1
(43) Pub. Date: Jun. 2, 2022

(54) VIRAL VECTOR PURIFICATION APPARATUS AND METHOD

(71) Applicant: GLOBAL LIFE SCIENCES SOLUTIONS USA LLC, MARLBOROUGH, MA (US)

(72) Inventors: VINCENT FRANCIS PIZZI, MILLIS, MA (US); JOSEPH MICHAEL MAKOWIECKI, OXFORD, MA (US)

(73) Assignee: GLOBAL LIFE SCIENCES SOLUTIONS USA LLC, MARLBOROUGH, MA (US)

(21) Appl. No.: 17/108,183

(22) Filed: Dec. 1, 2020

(51) Int. Cl. C12N 7/00; C12M 1/26

(52) U.S. Cl. CPC C12N 7/00; C12N 2750/14151; C12M 33/22

(57) ABSTRACT

A method for clarifying a bioprocess fluid comprising particles suspending in a cell culture fluid is provided. The method includes providing a cell culture suspended in an unclarified bioprocess fluid in a bioreactor. A chromatography affinity resin is added directly to the unclarified bioprocess fluid. The chromatography affinity resin binds a biological target. The unclarified bioprocess fluid with the bound biological target is passed into an assisted gravity settler.

### BACKGROUND

### Technical Field

Embodiments of the invention generally fall into the category of clarifying a bioprocess fluid and, in particular, the bulk purification of a viral vector from a cell culture.

### Discussion of Art

Viral vectors play a critical role in gene therapy and immunotherapy (e.g., chimeric antigen receptor T-cell immunotherapies, “CAR-T”) as carriers of genetic code modifiers and instructions. Common vectors can include, for example, retroviruses, lentiviruses, adenoviruses, adeno-associated viruses, plant viruses, and hybrid vectors. Viral vectors used in human treatments are commonly derived from viruses that naturally infect human or other mammalian cells.

In general, the production of viral vectors relies on a transfected host cell culture to produce new viral particles with the desired genetic contents contained within the viral vector. Cell cultures may be maintained as either adherent or suspension cell cultures. In broad terms, there are two modes of vector production: stable (continuous) cell production lines, and transient (inducible) production lines. Regardless of the chosen mode, once produced a usable viral vector must be separated and purified into a suitable final product. Due to the complexity of manufacturing viral vectors in bulk quantities, current Good Manufacturing Processes (“cGMPs” or “GMPs”) require that end-stage products have at least the attributes of being safe, correct identity, sufficient strength/potency over the shelf-life of the product, are free from contaminants, and are manufactured with monitored processes that incorporate sufficient quality control mechanisms.

Impurities may be derived from the host cell system within which the vector product is generated or from the downstream vector purification process. Potential sources of host cell related impurities can include: residual host cell proteins and nucleic acids derived from the production cells. Other impurities can include process-related residuals from the cell culture medium (i.e. bovine serum albumin) and downstream purification processes (i.e. detergents and chromatography resin components). The quantification and removal of host-cell impurities is important since certain host cell molecules can have toxic effects in the final drug product or can act as an adjuvant to stimulate an anti-vector immune response. Additionally, malformed or incomplete viral vector components (e.g., unfilled capsids, non-integrated viral proteins, viral peptides, or nucleic acids) are an additional source of impurities.

Production of viral vectors, such as in the production of Adeno Associated Virus (AAV) often require cell lysis as part of viral vector production. The lytic process introduces an impurity load over and above non-lytic processes. The debris of cellular components, DNA, cell membranes, etc. within the culture media, along with the viral vector target, must undergo a separation process in order to meet the standards above described for efficacy and purity. Traditional separation and purification processes adversely impact the overall yield of viral vector obtained from the production process; this results in increased manufacturing complexity and increased cost of the final product.

Biopharmaceutical production is trending toward higher cell densities and product titers such that single-use harvest systems are becoming financially and logistically advantageous. Single-use bioreactors for cell culture volumes greater than or equal to 2,000 L provide an economically attractive alternative to stainless steel infrastructure as batch production titers continue to increase. Many biopharmaceuticals, including viral vectors, are initially separated from producer cells in a crude harvest step prior to downstream purification via chromatography systems. Volumetrically scalable solutions for this harvest step include centrifugation and/or depth filtration when a protein or other product (e.g., virus) is produced.

Depth filtration is an example single-use harvest method to remove intact cells and cellular debris via primary and secondary clarification, respectively. The depth filtration process suffers from cell caking and clogging as bioreactor cell densities gradually increase, which is undesirable for manufacturing productivity. Further, the total filtration area of depth filtration tends to scale proportionally with cell density for primary harvest, which is undesirable for inventory floor space and is technically and economically prohibitive at cell densities greater than 30 million cells mL⁻¹. While centrifugation may be a suitable alternative for large fixed-asset (stainless steel) manufacturing sites, it may be prohibitive in smaller single-use contexts due to capital equipment expenditure, sterilization preparation time between batches, and centrifugation equipment maintenance. Also, centrifugation-based harvest may suffer from unsatisfactory product loss when bioreactor feedstocks contain high cell densities (e.g. solids exceeding 10% of the culture mass). Past attempts to address cell separation typically employ inclination that includes vertically flowing cell containing fluid at an angle between 30° and 80° from horizontal toward a separation channel. Cell separation is transverse to the vertical fluid flow through separation channel for cells to flow into a separate chamber. Separation is limited to the cells passing over the separation channel amounting to a filtration device, prone to fouling, for perfusion operations with flow rates below 40 L d⁻¹, which is not applicable to batch cell culture primary clarification operations.

Process impurities are usually present in trace amounts, but it is important that they meet pre-set safety guidelines. Example process impurities can include residual solvents, detergents, buffers, or other undesirable compounds that are Mass Spectrometry (MS) and chromatography methods are widely used to identify detergents and organic solvents in the vector preparation.

Thus, in light of the above, there is needed a method of purifying viral vectors in a large scale production setting that increases yield while minimizing overall process complexity.

### BRIEF SUMMARY OF THE INVENTION

In an embodiment, an apparatus for the clarification of a bioprocess fluid is provided. The apparatus includes a bioreactor operatively coupled to an assisted gravity settler configured to receive a volume of unclarified bioprocess fluid from the bioreactor.

In another embodiment, a method for clarifying a bioprocess fluid comprising particles suspending in a cell culture fluid is provided. The method includes providing a cell culture suspended in an unclarified bioprocess fluid in a bioreactor. A chromatography affinity resin is added directly to the unclarified bioprocess fluid. The chromatography affinity resin binds a biological target. The unclarified bioprocess fluid with the bound biological target on the resin is passed into an assisted gravity settler.

In another embodiment, a method of clarifying adeno associated virus (AAV) is provided. The method includes providing a cell culture transfected to produce adeno associated virus (AAV) resulting in an unclarified bioprocess fluid containing AAV in a bioreactor. A chromatography affinity resin is added directly to the unclarified bioprocess fluid. The AAV is subsequently bound to the chromatography affinity resin. The unclarified bioprocess fluid with the bound AAV on the resin is passed into an assisted gravity settler.

In yet another embodiment, a method of clarifying a viral vector is provided. The method includes providing a bioreactor with a cell culture capable of transfection for production of a viral vector in a bioprocess fluid in a bioreactor. A vector for encoding a viral vector is then introduced into the cell culture. Viral vector production is then initiated. Bioprocess fluid containing the viral vector is removed from the cell culture. Fresh bioprocess fluid is introduced to the cell culture in a continuous process. The viral vector containing bioprocess fluid is then processed with at least an assisted gravity settler.

### BRIEF DESCRIPTION OF THE DRAWINGS

FIG. 1 is an example of a process schematic according to an embodiment of the invention.

FIG. 2 is a schematic of a method according to an embodiment of the invention.

### DETAILED DESCRIPTION OF THE INVENTION

Unless otherwise defined, all technical and scientific terms used herein possess the meaning commonly understood by the skilled artisan. In the case of inconsistencies, the present disclosure, including definitions, controls.

As used above, and throughout the description, the following terms, unless otherwise indicated, shall be understood to have the following meanings:

As used herein, “about” means within 10%, such as within 5% and further such as within 2.5%, of a given value or range. When the term “about” is used in conjunction with a numerical range, it modifies that range by extending the boundaries above and below the numerical values set forth. The term “about” may also indicate reasonable tolerances and variations reflected in preparations and compositions under manufacturing processes.

As used herein, “bioreactor” refers to an apparatus used for the growth and sustenance of biological material as commonly used. The term encompasses all associated equipment necessary to sustain the material growth (e.g., a growth vessel/chamber; process variable measurement and monitoring equipment; pumps; lines; and the like). A “bioreactor” may be as simple as a shake flask with a cell culture or as complicated as a multi-story stainless-steel large volume manufacturing system.

As used herein the term “vector” is used as commonly understood in molecular biology (e.g.; plasmids, phages, cosmids, bacterial artificial chromosomes, yeast artificial chromosomes, and human artificial chromosomes) and is distinguished from “viral vectors” which employ a virus to deliver a vector. In essence a “vector” in any form is a means of transporting biological information/components to effect change in a biological system. By way of non-limiting example, adeno-associated virus (AAV) vector-based gene therapy utilizes AAV particles to transport genetic material for insertion into a targeted chromosome.

As used herein the term “cell culture” is used as commonly understood to include not just the cells themselves, but the media (“cell culture media” or “media”) supplying nutrients and/or support to the cultured cells.

Also as used herein the term “bioprocess” is a specific process that uses complete living cells, or their components, to obtain desired products. A bioprocess may, in general, be divided into three stages or phases: preparation, production, purification. A “bioprocess fluid,” likewise, is one used as part of the bioprocess. The composition of a “bioprocess fluid” may change over the course of time. By way of non-limiting example, a bioprocess fluid may be cell culture media with nutrients, pH buffers, waste products, and biological targets. A bioprocess may further be characterized as a “batch” or “continuous” process. In a “batch” process a single production and harvest is contemplated. In a “continuous” process multiple production and/or harvest steps may occur and bioprocess fluids may be removed and replaced from and/or to a bioreactor.

As used herein the term “biotherapeutically active” is a product that alters a chemical or physiological function of a cell, tissue, organ, or organism. A biotherapeutically active product may be formed from: cells, proteins, viruses, vaccines, DNA, RNA, peptides, small or large molecules, or combinations or parts thereof.

As used herein the term “biological target” refers to a product of interest produced in a bioprocess. By way of non-limiting example, AAV particles may be biological targets of interest in bioprocess utilizing a cell culture to produce the AAV particles. Other biological targets may include therapeutic proteins, polysaccharides, vaccine components, small molecules, and other biologics.

As used herein the term “affinity chromatography resin” is a chromatographic stationary phase that exploits molecular properties (e.g., charge, hydrogen bonding, ionic interaction, disulfide bridges, hydrophobic interaction, etc.) In some instances the chromatography affinity resin may be cross-linked 6% agarose matrix with a polysaccharide polymer bound to a ligand. In other instances, the chromatography affinity resin may be AVB Sepharose®. In still other instances the affinity chromatography resin may be poly(styerine-divinylbenzene) backbone based beads roughly 50 microns in diameter such as found in CaptureSelect® resins. Other chromatographic resins, such as those that purify based upon molecular size, are also contemplated.

As used herein the term “assisted gravity settler” refers to a separation device configured to receive the flow of a bioprocess fluid from a bioreactor and to separate at least a portion of particles from the from the bioprocess fluid to generate a substantially clarified bioprocess fluid. An example may be found at PCT Pub. No: WO2020/052996 the entirety of which is incorporated by reference.

In some embodiments, an assisted gravity settler aids in the clarification of a bioprocess fluid containing suspended particles by flowing the unclarified bioprocess fluid from a fluidically coupled bioreactor through a plurality of mesofluidic channels within the separation device. In certain embodiments the mesofluidic channels may be substantially parallel to each other and may be within 2-20 mm in height. The residence time of the bioprocess fluid within the separation device may range from 10-40 minutes relative to the time at which all or a portion of the fluid first enters the device. The assisted gravity settler may be used in either batch or continuous processes. One or more additional purification subsystems may be fluidically coupled to one or more outlets of the assisted gravity settler for further processing of the clarified bioprocess fluid. The additional purification subsystems can include chromatographic separation devices, secondary depth filtration, a polishing membrane, or any combination thereof. The assisted gravity settler may include one or more additional inlets and outlets for the introduction and/or removal of buffers, flushing fluids, fixers, or other compounds as required.

In certain embodiments the assisted gravity settler may operate at an angle of less than or equal to 15°, relative to a working surface, and residence time of less than or equal to 25 minutes. The exact timing and angles may be adjusted by those of skill in the art to account for process variables without departing from the scope of the invention. Residence times of 5-45 minutes also possible as are angles within a range of 5-45°. In some embodiments, the angle used may be an angle between substantially 0°-30°, or an angle between substantially 0°-10°, such as 10°, 5°, or about 0° (e.g., 0°±5°). In contrast, inclined settlers are dependent upon the Boycott effect, which may require operation angles around 30° or greater to achieve sedimentation. In some embodiments, the device may be positioned at the angle throughout the separation process. However, in some embodiments, the device may be intermittently or periodically tilted from a first angle to a second or more angles. Additional angles may evacuate air from the mesofluidic channels to increase separation efficiency of the device.

In alternative configurations, the assisted gravity settler may include any suitable quantity of fluid inlets, fluid inlet manifolds, fluid outlets, fluid outlet manifolds, and may or may not include a lateral inlet channel and/or a lateral outlet channel. Varying amounts of fluid inlets and fluid outlets, as well as fluid inlet manifolds and fluid outlet manifolds, may allow customization or selection of a pressure drop across the device, and thus, may vary the flow rate of the cell culture fluid through the device. This may allow for customization of the device based on a target application. A fluidic path between the fluid inlet and the fluid outlet of the assisted gravity settler device may be unidirectional in a linear or serpentine configuration. Additionally, inclusion of a lateral inlet channel and/or a lateral outlet channel may provide for minimization of the profile of the device while still allowing for substantially equal distribution of the cell culture fluid at a particular flow rate through the device.

In operation, a cell culture fluid may be provided to the assisted gravity settler at a particular flow rate. This is the flow rate that the cell culture fluid passes through the mesofluidic channels within the device. The cell culture fluid may enter a fluid inlet manifold and may be distributed substantially evenly between multiple mesofluidic channels. As the cell culture fluid traverses the mesofluidic channels, a density difference between the particles contained in the cell culture fluid (e.g., cells and/or bound viral vectors) and the surrounding fluid of the cell culture fluid may cause the particles to settle and collect on a lower interior surface of each mesofluidic channel. Settling of the particles of the cell culture fluid on the lower interior surface of the mesofluidic channels may be further caused by a separation force acting on the higher density particles within the cell culture fluid. The separation force may be an ambient gravitational force, such that no separate or additional force is needed to cause settling of the particles within the mesofluidic channels. Settling of the particles of the cell culture fluid within the mesofluidic channels as the cell culture fluid flows through the device may yield a substantially clarified fluid layer (e.g. >80% particle removal) of the cell culture fluid that can be recovered as an output via a fluid outlet. As such, a product, such as a protein, of the biopharmaceutical process within the fluid layer of the cell culture fluid may be recovered.

In certain embodiments, the separation of a viral vector is accomplished through the binding of the vector to a chromatography affinity gel. The gel with the bound agent is settled out of the bioprocess fluid through the use of the assisted gravity settler. The settled gel may be washed of debris one or more times and the bound viral vector product eventually eluted either as a final step or for subsequent processing/polishing steps. One or more assisted gravity settler devices may be arranged in series or in parallel to accommodate varying volumes and/or continuous vs. batch processes.

As used herein “buffer” or “buffers” denotes process liquids used as a part of the manufacturing cycle. One or more fluids may be combined to form a buffer. It is not necessary for a buffer to actually buffer pH or another ion although such buffers may fall under this definition. Example buffers may include: water (water-for-injection (WFI) quality, cell culture, and molecular biology grade); buffered salines and balanced salts (DPBS, PBS, HBSS, EBSS); chromatography buffer solutions; or cleaning solutions (NaOH, WFI quality water, 20% alcohol).

Embodiments of the invention involve the separation of a biological target form the debris field of a bioprocess fluid by first adding chromatography affinity resin directly to a bioprocess fluid, binding the biological target, and then passing bioprocessing fluid to an assisted gravity settler. Cell debris flow out of the settler and the chromatography affinity resin bound with the biological target is captured in the assisted gravity settler device. The captured resin is processed with buffer washing and elution of the biological target. Subsequent purification and/or packaging steps, may be performed on the eluted material. The chromatography affinity resin may then be cleansed and re-used or disposed as appropriate.

While traditional methods to purify AAV post clarification have yields on the order of 30-38% due to depletion of the biological target at the clarification stage it is envisaged that those of ordinary skill in the art practicing embodiments of the instant invention will return higher yields above 38% since the chromatography affinity resin is added directly to the cell culture bioprocess fluid before clarification. Hence, the biological target is bound to the chromatography affinity resin before the bioprocess fluid is subsequently clarified and purified.

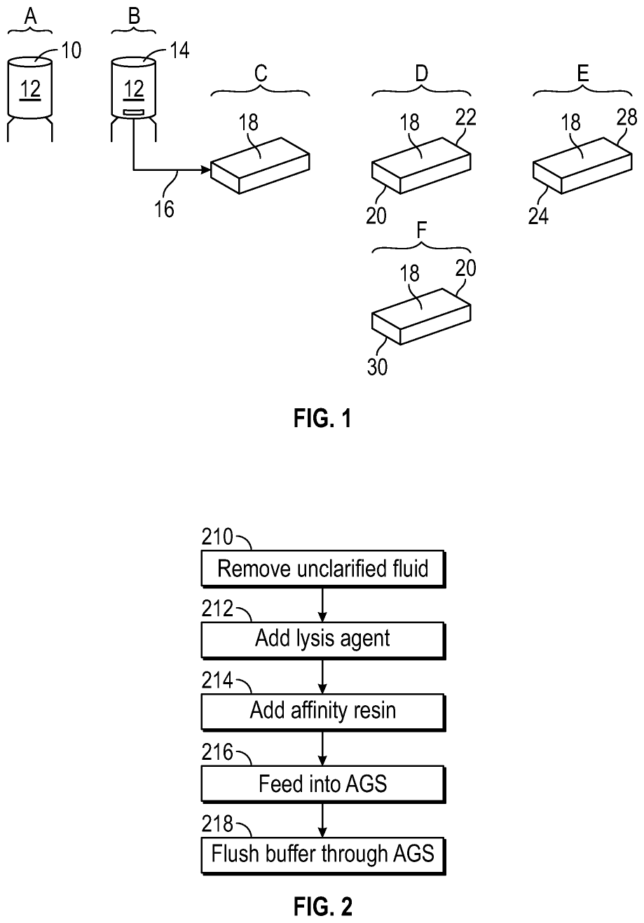

Turning to FIG. 1, a schematic representation of an embodiment of a method for performing the invention is represented. In an optional first step A, a cell lysis agent or other stimulus to modify the growth of the cell culture such as a detergent (ionic, non-ionic, or zwitterionic) and/or a nuclease (e.g., Benzonase Tx®) 10 are added to a bioreactor 12 containing host cells transfected to produce a biological target (AAV in this instance; in other embodiments a non-lytic virus, such as lentivirus, may be used) suspended in an unclarified bioprocess fluid. In a second step B, a chromatography affinity resin 14 (e.g., AVB Sepharose®) is added to the bioreactor binding the biological target. The amount of resin added is readily calculated by those of skill in the art without undue experimentation at least in view of the amount of product expected to be bound and the overall volumetric capacity of the bioprocess system. In third step C, the unclarified bioprocess fluid with the bound biological target is flowed 16 into an assisted gravity settler 18. The chromatography affinity resin with the bound biological target is captured within the assisted gravity settler 18. In a fourth step D, the assisted gravity settler 18 is buffer 20 flushed, removing cellular debris and other detritus as effluent 22, clarifying the bioprocess fluid. In a fifth step E, the biological target is then eluted from the chromatography affinity resin by passing an elution mixture 24 through the assisted gravity settler 18 resulting in eluent 28 with the biological target. In another optional step F, buffer 20 flow is then reversed through the assisted gravity settler 18 and the affinity chromatography resin (usually in bead form) is flushed 30 from the assisted gravity settler 18. The affinity chromatography resin may then be recovered for subsequent reuse or disposal. Flow rates, buffer compositions, fluid volumes, etc. are readily determined by those of skill in the art in view of overall system size, affinity chromatography gel composition, the type of viral vector produced and the standards of purity required.

Turning to FIG. 2, a schematic of a method according to an embodiment of the invention is presented. In a first step 210 an unclarified bioprocess fluid containing a mature cell culture producing a biological target may be removed from a bioreactor. In a second step 212 a cell lysis agent as above described, is then added to the removed unclarified bioprocess fluid. In a third step 214, a chromatography affinity resin is added to the unclarified bioprocess fluid. In a fourth step 216 the unclarified bioprocess fluid is fed into an assisted gravity settler. In a fourth step 218 a buffer is flushed through the assisted gravity settler removing cellular debris and other detritus as effluent, clarifying the bioprocess fluid. The remainder of the process is carried out as above described for FIG. 1. Those of ordinary skill in the art will appreciate that additional steps may be interposed or that certain steps may be rearranged in order of operation without departing from the broader scope of the disclosed invention.

In an embodiment, the pH of the bioprocess fluid is lowered before flowing the unclarified bioprocess fluid through the assisted gravity settler. In an embodiment, the pH may then be raised after buffer flush washing the captured chromatography affinity resin with the bound biological target. In other embodiments the concentration of a solubilized ion from a salt compound may be raised or lowered.

In an embodiment, the eluted biological target is passed to at least one secondary purification system (e.g., depth filtration, membrane filtration, chromatography, and/or centrifugation, etc.).

In an embodiment, a cell culture in a bioreactor suspended in a bioprocess fluid is transfected with a vector to produce a biological target. In certain embodiments the vector triggers the host cells to produce AAV altered to contain an genetic component for insertion into a target genome. A chromatography affinity resin (e.g., AVB Sepharose®) is added directly to the unclarified bioprocess fluid containing the biological target in the bioreactor binding the biological target to the chromatography affinity resin. The unclarified bioprocess fluid containing the biological target is then passed to the assisted gravity settler. As above described, the biological target is eluted from the chromatography affinity resin and passed to a secondary purification system.

In still other embodiments, biological target containing unclarified bioprocess fluid is removed from the bioreactor and fresh bioprocess fluid is provided to the bioreactor. The affinity chromatography resin is added to the biological target containing bioprocess fluid removed from the bioreactor, binding the biological target. The bound biological target containing bioprocess fluid is then processed with an assisted gravity settler. In some embodiments, the biological target is a viral vector.

The apparatus and methods described herein simplify manufacture of biologically produced biotherapeutic processes by eliminating one or more additional separation and clarification steps. The application of chromatography affinity resin directly to an unclarified bioprocess fluid significantly increases yield, particularly of AAV, as additional handling and washing steps that would otherwise degrade or remove a biological target are eliminated. Further, in performing embodiments of the invention, the decrease in equipment usage frees up manufacturing floor space increasing overall manufacturing plant production.

Finally, the written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. A method for clarifying a bioprocess fluid comprising particles suspending in a cell culture fluid, the method comprising the steps of:
providing a cell culture suspended in an unclarified bioprocess fluid in a bioreactor;
adding a chromatography affinity resin directly to the unclarified bioprocess fluid;
binding a biological target in the unclarified bioprocess fluid to the chromatography affinity resin; and
passing the unclarified bioprocess fluid with the bound biological target into an assisted gravity settler.

2. The method of claim 1 further comprising the step of :
adding a detergent and a nuclease to the unclarified bioprocess fluid before adding the chromatography affinity resin.

3. The method of claim 1, wherein:
the chromatography affinity resin further comprises a cross-linked 6% agarose matrix with a polysaccharide polymer bound to a ligand.

4. The method of claim 1, wherein:
the chromatography affinity resin is AVB Sepharose

5. The method of claim 1, further comprising the steps of:
capturing the chromatography affinity resin with the bound biological target within the assisted gravity settler;
buffer flush washing the captured chromatography affinity resin with the bound biological target;
eluting the biological target from the chromatography affinity resin;
reversing the buffer flow through the assisted gravity settler; and
reclaiming the chromatography affinity resin.

6. The method of claim 5 further comprising the steps of:
lowering the pH of the unclarified bioprocess fluid before flowing the unclarified bioprocess fluid through the assisted gravity settler; and,
raising the pH of the clarified bioprocess fluid after buffer flush washing the captured chromatography affinity resin with the bound biological target.

7. The method of claim 5 further comprising the step of:
passing the eluted biological target to at least one secondary purification system selected from the group of: depth filtration, membrane filtration, chromatography, and centrifugation.

8. The method of claim 1 wherein the bound biological target is at least one biotherapeutically active product selected from the group of: cells, proteins, viruses, vaccines, DNA, RNA, and peptides.

9. The method of claim 8 wherein the biotherapeutically active product is an adeno associated virus (AAV).

10. The method of claim 9 wherein the yield of AAV is (greater than or equal to 20%)

11. An apparatus for the clarification of a bioprocess fluid comprising:
a bioreactor; and
an assisted gravity settler fluidically connected to the bioreactor;
wherein the assisted gravity settler is configured to receive a volume of unclarified bioprocess fluid from the bioreactor.

12. The apparatus for the clarification of a bioprocess fluid of claim 11, wherein:
the unclarified bioprocess fluid contains a biological target bound to a chromatography affinity resin before it is received by the assisted gravity settler.

13. The apparatus for the clarification of a bioprocess fluid of claim 11 wherein:
the assisted gravity settler is further configured to pass an eluted biological target to at least one secondary purification system selected from the group of: depth filtration, membrane filtration, chromatography, and centrifugation.

14. The apparatus for the clarification of a bioprocess fluid of claim 11 wherein:
the bound biological target is at least one biotherapeutically active product selected from the group of: cells, proteins, viruses, vaccines, DNA, RNA, and peptides.

15. The apparatus for the clarification of a bioprocess fluid of claim 11 wherein the bound biological target is an AAV.

16. The apparatus for the clarification of a bioprocess fluid of claim 11 wherein the chromatography affinity resin further comprises a cross-lined 6% agarose matrix with a polysaccharide polymer bound to a ligand.

17. The apparatus for the clarification of a bioprocess fluid of claim 11 wherein the chromatography affinity resin is AVB Sepharose.

18. A method of clarifying adeno associated virus (AAV), the method comprising:
providing a cell culture transfected to produce AAV resulting in an unclarified bioprocess fluid containing AAV in a bioreactor;
adding a chromatography affinity resin directly to the unclarified bioprocess fluid;
binding AAV to the chromatography affinity resin; and
passing the unclarified bioprocess fluid with the bound AAV into an assisted gravity settler.

19. The method of clarifying AAV according to claim 18, wherein:
the chromatography affinity resin is AVB Sepharose; and, the assisted gravity settler is further configured to pass an eluted AAV to at least one secondary purification system selected from the group of: depth filtration, membrane filtration, chromatography, and centrifugation.

20. A method of clarifying a viral vector, the method comprising:
providing a bioreactor;
providing a cell culture capable of transfection for production of a viral vector in a bioprocess fluid in the bioreactor;
providing a vector encoding for viral vector production to the cell culture;
initiating viral vector production;
removing viral vector containing bioprocess fluid from the cell culture;
providing fresh bioprocess fluid to the cell culture in a continuous process; and
processing the bound viral vector containing bioprocess fluid with an assisted gravity settler.
